(19) United States
(12) Patent Application Publication
(10) Pub. No.: US 2025/0235593 A1
(43) Pub. Date: Jul. 24, 2025

(54) DETERMINATION OF INTRAPERITONEAL VOLUME DURING PERITONEAL DIALYSIS

(71) Applicant: GAMBRO LUNDIA AB, Lund (SE)

(72) Inventors: Anders WIESLANDER, Lund (SE); Olof JANSSON, Vellinge (SE)

(21) Appl. No.: 18/855,578

(22) PCT Filed: Apr. 5, 2023
(86) PCT No.: PCT/EP2023/058909

§ 371 Date: Oct. 9, 2024

(30) Foreign Application Priority Data
Apr. 14, 2022 (SE) 2250461-7

(51) Int. Cl. A61M 1/28; G16H 40/63

(52) U.S. Cl. CPC A61M 1/281; G16H 40/63; A61M 2202/0007; A61M 2202/0014; A61M 2202/04; A61M 2205/3327; A61M 2205/3379; A61M 2205/50; A61M 2210/1017

(57) ABSTRACT

A system for peritoneal dialysis is operated, according to a measurement method, to supply a first fluid to a peritoneal cavity, PC, extract a first amount of fluid from the PC while leaving an intraperitoneal amount of fluid in the PC, and measure a first value of a concentration-related parameter of the thus-extracted fluid. The system is then operated to supply a second amount of a second fluid to form a mixture with the intraperitoneal amount of fluid in the PC, extract a third amount of the mixture from the PC, and measure a third value of the parameter of the thus-extracted mixture. The second fluid has a second value of the parameter and differs in composition from the first fluid. The use of such a second fluid enables the intraperitoneal amount to be determined, with high accuracy, based on the second amount and the first, second and third values.

### TECHNICAL FIELD

The present disclosure relates generally to peritoneal dialysis, and in particular to a technique of determining or estimating the amount of fluid in the peritoneal cavity during peritoneal dialysis.

### BACKGROUND ART

In the treatment of individuals suffering from acute or chronic renal insufficiency, dialysis therapy may be needed. One category of dialysis therapy is peritoneal dialysis (PD). In PD, a treatment fluid (“dialysis fluid”) is infused into the individual's peritoneal cavity, also known as abdominal cavity. This cavity is lined by a peritoneal membrane (“peritoneum”) which is highly vascularized. Substances are removed from the patient's blood mainly by diffusion across the peritoneum into the treatment fluid. Excess fluid (water) is also removed by osmosis induced by the treatment fluid being hypertonic.

In automated peritoneal dialysis (APD), the dialysis treatment is controlled by a machine, commonly known as a “cycler”. The machine is connected in fluid communication with the peritoneal cavity and is operated to control the flow of fresh dialysis fluid into the peritoneal cavity and the flow of spent dialysis fluid from the peritoneal cavity. Common complications in APD are so-called overfill and underfill. Overfill refers to inadvertent presence of an excessive fluid volume in the peritoneal cavity. Conversely, underfill refers to provision of an unexpectedly small fluid volume in the peritoneal cavity. Overfill may cause the patient to experience severe pain and may even be fatal, as noted in the article “Drain pain, overfill, and how they are connected”, by Peter Blanke, published in Peritoneal Dialysis International, Vol. 34, pp. 342-344 (2014). Underfill may lead to poor efficiency of the PD treatment and may also result in pain if it causes the machine to draw the peritoneal cavity completely empty of fluid.

The risks of underfill and overfill may be mitigated if the actual amount of fluid in the peritoneal cavity is known at one or more time points during PD treatment. The actual amount of fluid is commonly referred to as “intraperitoneal volume”, abbreviated IPV. Further, knowledge of the IPV when the spent dialysis fluid has been expelled from the peritoneal cavity, known as “residual volume”, may also be used to anticipate the effectiveness of a PD treatment and help a clinician to (re-)configure the machine for a PD treatment.

EP2623139 proposes a procedure for estimating the residual volume in the peritoneal cavity during regular PD treatment. In the proposed procedure, the peritoneal cavity is drained of spent dialysis fluid at the end of a dwell phase, leaving an unknown residual volume in the peritoneal cavity, and the conductivity of the spent dialysis fluid is measured. The conductivity of fresh dialysis fluid is then measured, and a volume of fresh dialysis fluid is infused into the peritoneal cavity. Then, a small volume of fluid is extracted from the peritoneal cavity, and its conductivity is measured. By use of a dilution formula, the residual volume is calculated based on the infused volume of fresh dialysis fluid and the measured conductivities. While being a simple procedure, it seems to yield a poor accuracy of the calculated residual volume. Based on the numeric example given in EP2623139, an actual residual volume of 667 ml would be estimated with an error of ±98 ml, given as standard deviation. If the actual residual volume instead is 100 ml, the resulting error is ±66 ml. If a PD machine is configured based on an estimated residual volume at such poor accuracy, the resulting PD treatment may be suboptimal and the patient may still experience overfill or underfill.

### SUMMARY

It is an objective to at least partly overcome one or more limitations of the prior art.

One objective is to provide a technique that allows the intraperitoneal volume to be estimated during peritoneal dialysis and at an improved accuracy.

One or more of these objectives, as well as further objectives that may appear from the description below, are at least partly achieved by a system for peritoneal dialysis, a computer-implemented method, and a computer-readable medium, embodiments thereof being defined by the dependent claims.

A first aspect is a system for peritoneal dialysis. The system comprises a fluid supply arrangement operable to convey fluid to and from a peritoneal cavity, a sensor arrangement operable to measure a concentration-related parameter, and a control arrangement which is connected to the fluid supply arrangement and the sensor arrangement. The system is configured to operate the fluid supply arrangement to supply a first fluid to the peritoneal cavity; operate, after supplying the first fluid, the fluid supply arrangement to extract a first amount of fluid from the peritoneal cavity so as to leave an intraperitoneal amount of fluid in the peritoneal cavity; and obtain, from the sensor arrangement, a first value of a concentration-related parameter of the thus-extracted fluid. The system is further configured to: operate the fluid supply arrangement to supply a second amount of a second fluid to the peritoneal cavity. The second fluid has a second value of the concentration-related parameter and forms a mixture with the intraperitoneal amount of fluid in the peritoneal cavity, wherein the first and second fluids differ in composition. The system is further configured to: operate the fluid supply arrangement to extract a third amount of the mixture from the peritoneal cavity; obtain, from the sensor arrangement, a third value of the concentration-related parameter of the thus-extracted mixture; and determine the intraperitoneal amount based on the second amount and the first, second and third values.

In some embodiments, the system is operated to achieve a difference between the second value and the first value that exceeds a threshold value, which is set to achieve a predefined accuracy of the intraperitoneal amount as determined based on the second amount and the first, second and third values.

In some embodiments, the threshold value is set to correspond to the second value being about 20%-75% larger than the first value or about 20%-75% smaller than the first value.

In some embodiments, the fluid supply arrangement is operable to generate the second fluid on demand, and wherein the control arrangement is configured to, based on first value, to operate the fluid supply arrangement to generate the second fluid to achieve said difference between the second value and the first value.

In some embodiments, the first fluid is a treatment fluid used in peritoneal dialysis therapy, and the second fluid is a dedicated test fluid for use in determining the intraperitoneal amount.

In some embodiments, the second fluid and the first fluid differ in concentration of at least one solute that affects the concentration-related parameter.

In some embodiments, solutes in the second fluid are the same as solutes in the first fluid.

In some embodiments, the second fluid has a different concentration of at least sodium compared to the first fluid.

In some embodiments, the second fluid has a different concentration of an osmotic agent compared to the first fluid.

In some embodiments, the second fluid has an osmolarity to minimize transfer of solvents through a peritoneal membrane in the peritoneal cavity.

In some embodiments, the second fluid has an osmolarity of 250-350 mOsm/l.

In some embodiments, the second value is smaller than the first value.

In some embodiments, the control arrangement is configured to perform a sequence of fluid exchange cycles, each comprising a fill phase, a dwell phase and a drain phase, wherein the sequence of fluid exchange cycles comprises a fluid exchange cycle, in which the fluid supply arrangement is operated to supply the first fluid to the peritoneal cavity in the fill phase and extract the first amount of fluid in the drain phase, and a consecutive fluid exchange cycle, in which the fluid supply arrangement is operated to supply the second amount of the second fluid to the peritoneal cavity in the fill phase and extract the third amount of the mixture during the drain phase, and wherein the control arrangement is configured to obtain the first value during the drain phase of the first cycle and to obtain the third value during the drain phase of the second cycle.

In some embodiments, the control arrangement is configured to operate the fluid supply arrangement so that the first amount is 25%-95% of an estimated total amount of fluid present in the peritoneal cavity.

In some embodiments, the control arrangement is configured to operate the fluid supply arrangement so that the second amount is 25%-100% of the first amount.

In some embodiments, the control arrangement is configured to determine the intraperitoneal amount by use of a dilution formula, which is given by IPV1=V2·(C3−C2)/(C1−C3), wherein V2 is the second amount, C1 is the first value, C2 is the second value, and C3 is the third value.

In some embodiments, the control arrangement is configured to determine the intraperitoneal amount during extraction of the third amount and terminate the extraction of the third amount based on the determined intraperitoneal amount and the second amount.

In some embodiments, the control arrangement is configured to terminate the extraction of the third amount to attain a predefined difference between the third amount and a sum of the determined intraperitoneal amount and the second amount.

A second aspect is a computer-implemented method of operating a system for peritoneal dialysis. The method operates the system to supply a first fluid to a peritoneal cavity; extract, after supplying the first fluid, a first amount of fluid from the peritoneal cavity so as to leave an intraperitoneal amount of fluid in the peritoneal cavity; and measure a first value of a concentration-related parameter of the thus-extracted fluid. The method further operates the system to supply a second amount of a second fluid to the peritoneal cavity. The second fluid has a second value of the concentration-related parameter and forms a mixture with the intraperitoneal amount of fluid in peritoneal cavity, wherein the first and second fluids differ in composition. The method further operates the system to extract a third amount of the mixture from the peritoneal cavity; measure a third value of the concentration-related parameter of the thus-extracted mixture, and determine the intraperitoneal amount based on the second amount and the first, second and third values.

A third aspect is a computer-readable medium comprising computer instructions which, when executed by one or more processors, cause the one or more processors to perform the method of the second aspect.

The measurement technique as defined by the foregoing aspects provides the technical advantage of allowing for a significant improvement in the accuracy of the estimated intraperitoneal volume, by virtue of the second fluid having a different composition than the first fluid. The ability to measure intraperitoneal volume requires a difference in the concentration-related parameter (for example, conductivity) between the second fluid and the fluid to which it is added in the peritoneal cavity, i.e. between the second and first values. If the same fluid is used as both first fluid and second fluid, as proposed in the prior art, this difference is basically fixed since it is mainly caused by the dilution of the first fluid by ultrafiltrate while the first fluid resides in the peritoneal cavity in a dwell phase. By using a second fluid that differs in composition from the first fluid, this difference is controllable and may be set to achieve a desired accuracy. The technique of the foregoing aspects also makes it possible to measure the intraperitoneal volume at any time before, during or after PD therapy, not only after completion of a dwell phase as in the prior art.

Still other objectives, aspects, embodiments and technical effects, as well as features and advantages may appear from the following detailed description, from the attached claims as well as from the drawings. It may be noted that any embodiment of the first aspect, as found herein, may be adapted and implemented as an embodiment of the second and third aspects.

### BRIEF DESCRIPTION OF THE DRAWINGS

FIG. 1 shows an example system for automated peritoneal dialysis (APD).

FIG. 2 is an example plot of intraperitoneal volume versus time during a sequence of fluid exchange cycles in APD therapy.

FIG. 3 is a flow chart of an example method of determining an intraperitoneal amount of fluid in the peritoneal cavity.

FIG. 4 illustrates the fill state of the peritoneal cavity during the method in FIG. 3.

FIG. 5 is a graph of estimated error in intraperitoneal volume as a function of a ratio of first and second concentration-related values of fluids present in the peritoneal cavity during the method in FIG. 3.

FIGS. 6-7 show a system for APD configurable to perform the method of FIG. 3.

FIG. 8 is a block diagram of an example control arrangement.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

Like reference signs refer to like elements throughout.

The present disclosure relates to a technique of estimating the amount of fluid in the peritoneal cavity of a patient during peritoneal dialysis (PD). In the following, this amount is denoted “intraperitoneal volume”, IPV. The peritoneal cavity is located in the patient's abdomen and lined by a peritoneal membrane, which is composed of a parietal peritoneum and a visceral peritoneum. PD uses the peritoneal membrane to exchange fluid and dissolved substances between a treatment fluid, present in the peritoneal cavity, and the blood of the patient. PD may be employed to remove excess fluid, correct electrolyte problems, and remove toxins. In PD, the treatment fluid (“PD fluid”) is first introduced into the peritoneal cavity and then removed, in accordance with a predefined cycling schedule. The PD fluid typically comprises electrolytes and an osmotic agent. The osmotic agent is a substance dissolved in water and capable of driving a net movement of water by osmosis across the peritoneal membrane due to concentration differences of the osmotic agent on each side of the membrane. The osmotic agent may, for example, comprise one or more of glucose (aka dextrose), L-carnitine, glycerol, icodextrin, fructose, sorbitol, mannitol or xylitol.

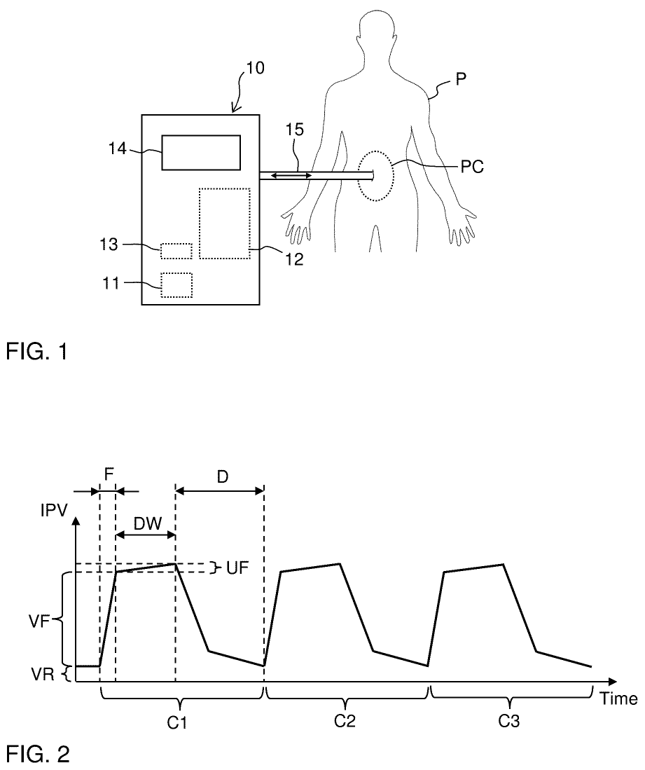

FIG. 1 schematically depicts an automated PD system 10, which is configured to perform PD therapy in relation to a patient P. The PD system 10 is also configured to estimate the intraperitoneal volume, IPV, in accordance with a technique described hereinbelow. The PD system 10 comprises a control arrangement 11, which is configured to control the operation of the system 10 and calculate the IPV. A fluid supply arrangement, FSA, 12 is operable to provide PD fluid for use in the PD therapy, as well as a dedicated test fluid for use in determining the IPV (below). The PD system 10 further comprises a sensor arrangement 13, which is configured to measure a concentration-related parameter of a fluid, such as the PD fluid or the test fluid. For brevity, the concentration-related parameter is denoted CRP in the following. The CRP is representative of the relative or absolute amount of one or more substances in the fluid. For example, the CRP may be given by one or more values of conductivity, resistivity, or concentration. In the example of concentration, the CRP may represent sodium and/or an osmotic agent, such as glucose. The sensor arrangement 13 may comprise more than one physical sensor unit. For example, separate sensor units may be provided to measure the CRP for the PD fluid and the test fluid, respectively. Alternatively or additionally, sensor units of different types may be provided to measure different CRPs.

The PD system 10 in FIG. 1 further comprises a user interface, UI, 14 which is configured to enable user interaction with the system 10. The user may be the patient P or a caretaker. The term “user interface” is intended to include any and all devices that are capable of performing guided human-machine interaction. The UI 14 may be configured to present operational data, for example current settings of the system, the progress of the PD therapy, current IPV, user instructions, alerts, and warnings. The UI 14 may be further operable to allow the user to enter data into the system 10, for example settings or instructions. The UI 14 may comprise a combination of a presentation device and data entry hardware. The presentation device may comprise a display and/or or a loudspeaker. The data entry hardware may include one or more of a keyboard, keypad, computer mouse, control buttons, touch panel, microphone and voice control functionality, camera and gesture control functionality, etc. In one implementation, the UI 14 is or comprises a touch-sensitive display, also known as touch screen.

As seen in FIG. 1, the PD system 10 further comprises a fluid connection device 15, which is coupled to the peritoneal cavity, PC, of a patient P. The fluid connection device 15 may be a tubing or the like, which is connected to an implanted catheter (not shown) in fluid communication with the PC. As indicated by a double-ended arrow, the PD system 10 is operable to convey fluid into and out of the PC through the fluid connection device 15.

The PD system 10 may be configured for any type of automated PD (APD) therapy, including but not limited to Continuous Cyclic PD (CCPD), Intermittent PD (IPD), Tidal PD (TPD), or Continuous Flow PD (CFPD). CCPD is also known as conventional APD. A typical cycling scheme of CCPD consists of three to five exchanges of PD fluid during the night. During daytime, a volume of PD fluid may or may not be left in the PC. IPD consists of frequent cycles performed over 8-10 hours per session, three times weekly. In IPD, the PC is typically drained and “dry” between sessions. TPD consists of an initial infusion of PD fluid followed by a variable dwell and partial drain of the PD fluid, leaving a residual volume in the PC until a final drain. CFPD is a continuous therapy that involves filling the PC with a desired volume of PD fluid, whereupon the in- and outflows of PD fluid are balanced.

FIG. 2 is a graph of IPV as a function of time during an example APD session, which comprises three consecutive exchange cycles C1-C3 and in which the PC is drained at the end of the session. At the start of the session, the patient has a residual volume VR in the PC. Each exchange cycle comprises a fill phase F, a dwell phase DW and a drain phase D, as shown for the first exchange cycle C1. In the first exchange cycle C1, an amount VF of PD fluid is infused into the PC. During the phases F, DW and D, fluid is transported through the peritoneal membrane, a process known as ultrafiltration (UF). Depending on the osmotic pressure gradient both positive UF and negative UF is possible. During an exchange cycle, the accumulated effect of UF is normally an increase in fluid volume, as indicated in FIG. 2. For illustration purposes, the effect of UF is only indicated for the dwell phase in FIG. 2. During the drain phase, spent PD fluid is extracted from the PC, leaving a residual volume. This residual volume may differ from the initial residual volume and also between cycles C1-C3.

It should be understood that the graph in FIG. 2 is conceptual and given under the assumption that fill volumes, drain volumes, durations, etc. are identical between exchange cycles. In practice, this need not be the case.

In PD, UF is normally quantified as the difference between drained volume and infused volume. Drained volume may vary considerably between cycles, and calculation of UF, by drained volume, may be averaged for several cycles on a daily or weekly basis. A good estimate of UF and knowledge of the patient's weight are key factors for achieving an adequate dialysis treatment. However, the IPV at any given time point in not known. The IPV may be indirectly estimated using computer simulations if the residual volume (VR), the infused volume (VF) and characteristics of the peritoneal membrane are known. As understood from FIG. 2, the IPV changes over time. By knowing the IPV, it is possible to optimize PD therapy. For example, it is possible to avoid overfilling the PC in the fill phase F or dwell phase DW. Overfilling is a risk for the patient and should be avoided. The risk of overfill may be aggravated in some modalities of APD, such as TPD. By knowing the IPV, it is also possible to adjust the residual volume, if desired. For example, if IPV is known, the drain phase D may be controlled to leave a smaller fluid volume in the PC after the drain phase D. By this reduction in residual volume, it is possible to increase the infused volume VF in the next fill phase F (cf. FIG. 2). The increase in VF results in an increased amount of fresh PD fluid in the PC during the dwell phase DW, and may also provide a larger contact surface between the PD fluid and the peritoneal membrane in the PC, leading to improved treatment efficiency. Further, knowledge of the IPV may be used to determine an optimal dwell time, determine membrane properties and the exchange of different solutes. Still further, knowledge of the IPV may be helpful in detecting a dislocated or malfunctioning catheter. Knowledge of the IPV may also be used in the drain phase D to mitigate the risk for so-called drain pain.

Conventionally, IPV is not regularly measured for patents on PD. Measurement of IPV is only performed when it is desired to exactly monitor UF and IPV, for example during clinical trials. Such measurement may use a PD fluid containing a macromolecular volume marker, making it possible to follow fluid volume changes intraperitoneally by monitoring the dilution of the volume marker. Examples of such volume markers include radioisotopically-labeled dextran (¹⁴C), radioactive albumin2 or hemoglobin. These procedures are way too complicated to be performed in connection with regular PD therapy in the home of the patient or in a clinic.

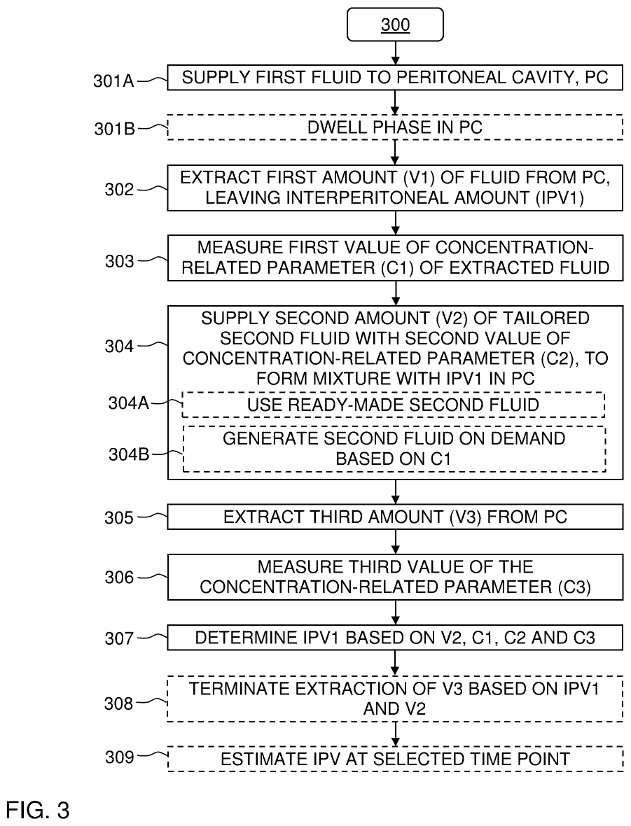

There is thus a need for a simple yet accurate technique of determining the IPV during PD therapy. The technique will be described in detail below, with reference to FIG. 3, and involves the use of a dedicated test fluid in addition to PD fluid.

In accordance with the present disclosure, the IPV is determined by measuring a concentration-related parameter (CRP) of the fluid in the peritoneal cavity before and after infusing a known amount of a test fluid into the peritoneal cavity. If the CRP is known also for the test fluid, the IPV may be calculated by use of a “dilution formula”. In its simplest form, the dilution formula is given by:

$$IPV = V 2 \cdot \frac{( C 3 - C 2 )}{( C 1 - C 3 )}$$

with C1 being the CRP of the fluid (“first fluid”) in the peritoneal cavity before infusion of the test fluid, C2 being the CRP of the test fluid (“second fluid”), V2 being the infused amount of test fluid, and C3 being the CRP of the fluid (“third fluid”) in the peritoneal cavity after infusion of the test fluid. By using a dedicated test fluid, it is possible to adjust C2 in relation to C1 to achieve a desired accuracy of the calculated IPV. It may be noted that the dilution formula may be expanded to account for other factors, such as the impact of ultrafiltration.

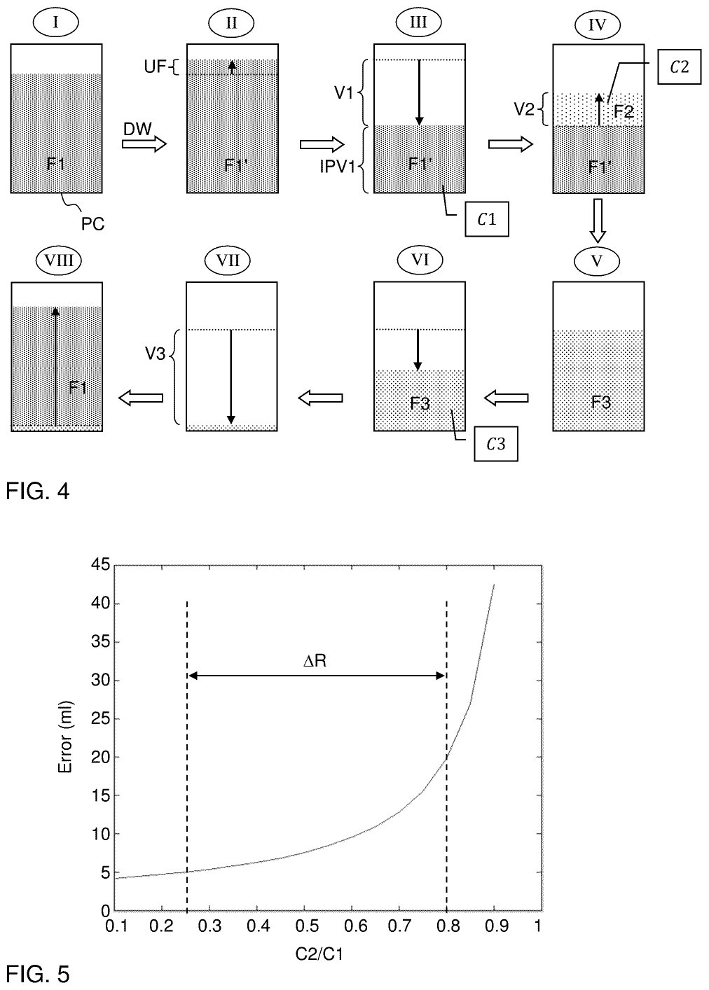

FIG. 3 is a flowchart of an example measurement method 300 for determining IPV. FIG. 3 will be presented with further reference to FIG. 4, which illustrates the fill state of the peritoneal cavity, PC, at different stages during the method 300. The method 300 may be performed by the system 10 in FIG. 1. In the following description, it is presumed that the volumes of fluid infused into the PC and extracted from the PC are measured, for example by a flow meter, volumetric pumping, weighing, etc.

In step 301A, a first fluid is supplied to the peritoneal cavity, PC. This is shown at stage I in FIG. 4, in which the first fluid is represented as F1. The first fluid F1 may be a PD fluid with a composition for use in regular PD. As indicated by dashed lines, subsequent step 301B is optional. Step 301B corresponds to a dwell phase (cf. DW in FIG. 2), in which the first fluid F1 resides in the PC and solutes and/or water are exchanged through the peritoneal membrane. The dwell phase is performed between stages I and II in FIG. 4 and may result in an increase in the amount of fluid in the PC by ultrafiltration, UF. During the dwell phase, the composition of the first fluid F1 is changed. The fluid in the PC at the end of the dwell phase is denoted “modified first fluid” and is represented as F1′ in FIG. 4. In step 302, a first amount (V1) of fluid is extracted from the PC to leave an unknown first intraperitoneal amount, IPV1, in the PC. If step 301B is omitted, F1 is extracted in step 302. If step 301B is included, F1′ is extracted in step 302, as shown at stage III in FIG. 4. In step 303, the CRP of the extracted fluid is measured, by use of the sensor arrangement 13 (FIG. 1), resulting in a CRP value C1. Step 303 may be performed at one or more time points during step 302. In step 304, a second amount (V2) of a dedicated test fluid (“second fluid”) is supplied to form a mixture with the first intraperitoneal amount IPV1 in the PC. The supply of the test fluid, represented as F2, is shown at stage IV in FIG. 4, and the resulting mixture is shown at stage V and is represented as F3. The test fluid F2 differs from the first fluid (PD fluid) F1. Specifically, the test fluid and the PD fluid differ in composition and have different values of the CRP. The CRP value of the test fluid is designated C2 herein and may be a predefined value or be measured by use of the sensor arrangement 13 (FIG. 1). For example, if the test fluid is generated on-demand, its CRP value may be verified by use of the sensor arrangement 13 before the test fluid is supplied to the PC. The use of a dedicated test fluid makes it possible to control the accuracy of IPV1, as calculated in step 307 (below), by proper selection of C2 in relation to C1.

The mixing of F1′ and F2 into F3 may be achieved by waiting for a predefined time period. Alternatively or additionally, mixing may be promoted by appropriate design of the implanted catheter and/or by agitation of the fluid in the PC, for example by the patient moving around or being moved, for example rolled from side to side. In step 305, assuming that the mixture F3 has been formed, a third amount (V3) is extracted from the PC. The third amount may be given by a predefined value, or be dynamically determined (cf. step 308 below). The extraction of V3 is shown at stages VI-VII in FIG. 4. In step 306, the CRP of the extracted fluid (mixture F3) is measured, by use of the sensor arrangement 13, resulting in a CRP value C3. Step 306 may be performed at one or more time points during step 305. In step 307, IPV1 is determined based on V2, C1, C2 and C3, for example by calculation using the above-defined dilution formula.

Optionally, as shown by dashed lines, the method 300 may further comprise a step 308 of terminating the extraction of fluid from the PC, started in step 305, based on IPV1 and V2. If ultrafiltration is neglected, the amount of mixture F3 in the PC at start of step 305 is equal to the sum of IPV1 (stage III) and V2 (stage IV). Thus, the total amount of fluid at the onset of step 305 is known. In some embodiments, step 308 terminates the extraction to attain a predefined difference between V3 and the sum of IPV1 and V2 (“termination condition”). The predefined difference defines the residual amount of fluid in the PC at the termination. For example, as shown at stage VII in FIG. 4, V3 may be adjusted to effectively drain the PC of fluid. The ability to control the remaining amount of fluid in the PC at completion of step 305 gives the advantage of being able to maximize the amount of PD fluid that is infused into the PC in a subsequent fill phase, as shown at stage VIII in FIG. 4. This will in turn increase the efficiency of the PD therapy.

If it is detected, during the extraction initiated in step 305, that the desired amount of fluid cannot be withdrawn from the PC to trigger the termination step 308, the method 300 may comprise a step of instructing the user (patient or caretaker) to change the patient's position to thereby change the location of the fluid within the PC. For example, the patient may be instructed to sit up from a lying position. If this does not help, the extraction of fluid may be terminated.

It has been found that sensor data obtained from the sensor arrangement 13 in step 306, during the extraction of fluid initiated in step 305, may be analyzed to determine the degree of mixing between F2 and F1′ in the PC. Specifically, the time profile of CRP values measured by the sensor arrangement 13 during the extraction will represent the degree of mixing, at least as long as the impact of UF is small. If the degree of mixing is incomplete when the extraction is initiated, the CRP values are expected to stabilize at a value representative of complete mixing as the extraction of fluid continues. Correspondingly, it is possible to determine when C3, given by the sensor data, is sufficiently accurate. To improve accuracy, C3 may be calculated in step 306 as an average of at least a portion of the time profile of CRP values, for example a portion subsequent to a detected stabilization of the CRP values. It may be noted that also C1, and C2 if measured, may be determined by averaging of a measured time profile of CRP values.

It is also conceivable that step 306 is performed repeatedly for a respective current portion of the time profile, to generate a time sequence of C3 values based on the sensor data from the sensor arrangement 13, and that step 307 is also performed repeatedly to determine a corresponding time sequence of IPV1 values. By analogy with the C3 values, the IPV1 values are likely to be more and more accurate over time, as mixing improves in the PC while fluid is extracted. Step 308 may repeatedly update the termination condition based on the IPV1 values generated by such a step 307.

As shown by dashed lines, the method 300 may also comprise an optional step 309 of estimating IPV at a selected time point other than at the end of step 302 (stage III). By tracking the amounts of fluid infused into and extracted from the PC, and optionally by estimating ultrafiltration, IPV may be estimated at any time point based on the calculated value IPV1. It is thereby possible to quantitatively monitor IPV over time, for example to render a graph similar to FIG. 2 for one or more exchange cycles.

As noted above, the method 300 may involve a dwell phase (step 301B). Thereby, the method 300 may be performed during regular PD therapy, for example after any of the dwell phases indicated in FIG. 2. It is also conceivable to repeatedly perform the method 300 to calculate IPV1 after more than one dwell phase, for example every n:th dwell phase (for example, with n=1, 2 or 3). Such repeated execution of the method 300 will further improve the accuracy of IPV1. For example, repeated execution may reduce the impact of measurement uncertainty of the CRP values C1 and C3, as well the impact of inadequate mixing of F1′ and F2 on the accuracy of the CRP value C3.

It may also be noted that step 302 need not be performed after completion of a regular dwell phase during PD therapy. For example, step 302 may be initiated after partial completion of a dwell phase, or the dwell phase may be omitted altogether. In fact, the method 300 may be performed at any time point before, during or after PD therapy. Further, the method 300 is not restricted to the PD therapy represented in FIG. 2 but may be performed in conjunction with any modality of PD therapy, including but not limited to CCPD, IPD, TPD and CFPD. When IPV1 has been calculated, the IPV may be tracked during subsequent PD therapy (cf. step 309).

The outcome of the method 300 in FIG. 3 may be compared to the prior art technique described in the Background section, which uses only PD fluid and relies on the dilution of the PD fluid by ultrafiltration during a dwell phase. In respect of the above-defined dilution formula, the prior art technique is thereby limited in terms of the maximum achievable difference between C1 and C2, with C1 being the CRP of spent PD fluid and C2 being the CRP of the fresh PD fluid. This will in turn limit the minimum attainable error of the calculated residual volume according to the prior art technique. Using the values of measured conductivity in the only numerical example presented in EP2623139, the ratio C1/C2 is 0.96, which means that C1 is merely 4% smaller than C2. As noted in the background section, this yields an error of +98 ml in the estimation of the residual volume when the residual volume is 667 ml.

Generally, the use of a dedicated test fluid provides much greater flexibility in setting the difference between C1 and C2 to achieve a better accuracy of the calculated value IPV1.

For comparison, the magnitude of the error in calculated IPV1 has been simulated and the result is presented in FIG. 5. Before discussing FIG. 5, the assumptions behind the simulation will be briefly presented. The PD fluid and the test fluid are assumed to consist of NaCl, KCl, CaCl₂, MgCl₂, glucose and water. The fluid volume in the PC at the start of step 302 is assumed to be 2 liter, and the extracted amount of fluid in step 302 (V1) is assumed to be 1 liter. Thus, IPV1 is assumed to be nominally 1 liter. The infused amount of test fluid (V2) in step 304 is assumed to be 0.5 liter. Further, it is assumed that the fluid extracted in step 302 has an NaCl concentration of 110 mmol/l. This concentration is selected to yield a conductivity (C1) that matches the conductivity of an actual PD fluid after a dwell phase. The concentrations of NaCl and glucose in the test fluid are adjusted to achieve a given conductivity C2 and an osmolarity of 300 mOsm/l (see further below). Turning now to FIG. 5, the ordinate represents the error of IPV1, given as standard deviation, and the abscissa represents the ratio C2/C1, for C2/C1<1. The magnitude of the error has been calculated, for each ratio, by a Monte Carlo simulation. This simulation has thus been repeated for different values of C2 to yield an estimate of the error of the calculated IPV1 as a function of the ratio C2/C1. As shown, the error increases rapidly with increasing ratio C2/C1. In one example, it may be desirable to limit the error to ±20 ml, which occurs at a ratio C2/C1 of about 0.8, as indicated by a dashed line in FIG. 5. It may also be noted that a lower limit of C2/C1 may be given by considering potential health implications for the patient, for example by too low concentrations of one or more electrolytes and/or too high concentrations of glucose. In FIG. 5, such a lower limit is indicated at a ratio C2/C1 of about 0.25. Thus, as shown in FIG. 5, the operative range AR for C2/C1 is approximately 0.25-0.8, which corresponds to C2 being approximately 20%-75% smaller than C1. It may be noted that corresponding results are attainable by setting C2 larger than C1, i.e. C2/C1>1. Simulations indicate that the curve in FIG. 5 is effectively mirrored in relation to C2/C1=1. Thus, the operative range for C2/C1 is approximately 1.2-1.75, which corresponds to C2 being approximately 20%-75% larger than C1. One advantage of setting C2 smaller than C1 is that the test fluid will be less costly in terms of raw material. Although the simulations have been performed for a specific test fluid and for specific fluid volumes, the results are believed to be generally applicable.

Based on the foregoing discussion, it is realized that the method 300 may be implemented, by proper composition of the test fluid, to achieve a difference between C2 and C1 that exceeds a threshold value, which is set to achieve a predefined accuracy of IPV1 as determined by step 307. The difference may be given as a relative value or an absolute value. The predefined accuracy in terms of standard deviation may for example be ±50 ml, ±40 ml, ±30 ml or ±20 ml. As noted above, based on FIG. 5, to achieve a predefined accuracy of at least ±20 ml, the threshold value may be set to correspond to C2/C1=0.8 or less. This may be compared to C1/C2=0.96 as achieved by the prior art technique according to the numerical example in EP2623139.

Reverting to stage IV in FIG. 4, it may be desirable to limit the time period during which the test fluid F2 resides in the PC, to thereby limit any exchange of solutes and/or water through the peritoneal membrane that may be driven by the test fluid. However, a certain time period may be required to ensure sufficient mixing between the modified first fluid F1′ and the test fluid F2 in the PC, from stage IV to stage V in FIG. 4. If necessary, the dilution formula may be modified to compensate for the exchange of solutes and/or water caused by the test fluid.

Reverting to step 304 in FIG. 3, the test fluid may be either pre-manufactured (step 304A) or generated on demand (step 304B). If pre-manufactured, the test fluid may be a so-called ready-made fluid which may be delivered to the point-of-care in pre-filled bags. It is conceivable that the test fluid is available in different compositions, each with a different C2, and that step 304A is arranged to select one of the available compositions based on C1 given by step 303 to achieve an appropriate difference between C1 and C2. If generated on demand, the test fluid may be given any selected value of C2. Again, the selected value of C2 may be set in view of C1 to achieve a difference between C1 and C2 that yields an acceptable accuracy of IPV1. The on-demand generation may involve mixing of one or more concentrates with water and/or mixing one or more ready-made fluids.

In an alternative, the test fluid is obtained independent of C1, as measured in step 303, but is provided to have a C2 with a sufficient difference in relation to C1 for all realistic values of C1.

In some embodiments, the test fluid may have different composition for different patients, for example based on transport properties of the patient's peritoneal membrane. Such transport properties may be determined by a conventional peritoneal equilibration test (PET). For example, as known in the art, the peritoneal membrane may be classified into one of several transporter types depending on its transport properties. The method 300 may be implemented to use different test fluids for different transporter types. Alternatively or additionally, the method 300 may use different test fluids for different modalities of PD therapy. The different test fluids need not only differ by C2, but may also differ by osmolarity (below).

As noted above, the test fluid (second fluid) differs in composition from the PD fluid (first fluid). In some embodiments, this is implemented as a difference in concentration of at least one solute, which may be present in both the PD fluid and in the test fluid and which affects the CRP. In some embodiments, the at least one solute comprises sodium (Na). Such embodiments may be relevant when the CRP is conductivity (or equivalently, resistivity), since sodium has a profound impact on conductivity. Alternatively or additionally, the at least one solute may comprise an osmotic agent, for example glucose. Such embodiments may be relevant when the CRP is the concentration of the osmotic agent. For example, concentration sensors for glucose are commercially available at low cost. Alternatively or additionally, the at least one solute may include at least one of magnesium, calcium or lactate.

In some embodiments, the solutes in the test fluid F2 are the same as in the PD fluid F1. Such embodiments may be particular advantageous when the test fluid is generated on demand. For example, the test fluid may be generated as a diluted version of the PD fluid, either by diluting a pre-manufactured PD fluid by purified water, or by mixing one or more concentrates with water. In the latter example, the same concentrates may thus be used for generating both the PD fluid and the test fluid.

In some embodiments, the test fluid F2 is generated on demand by mixing two or more concentrates with water, and the value of C2 for the test fluid is adjusted by changing the amount of at least one of the concentrates. If the PD fluid F1 is generated on demand by mixing at least one concentrate with water, it is conceivable that both the PD fluid and the test fluid are generated by use of at least one common concentrate.

In some embodiments, the test fluid F2 is generated on demand by mixing two or more concentrates with water, of which one concentrate only contains the osmotic agent. Thereby, the osmolarity of the test fluid may be adjusted largely independent of conductivity.

In some embodiments, the test fluid F2 has an osmolarity to minimize transfer of solvents through the peritoneal membrane in the PC. As used herein, the term “osmolarity” is synonymous with osmotic concentration and is a measure of solute concentration, defined as the number of osmoles (Osm) of solute per unit volume of solution. An osmole is the number of moles of solute that contribute to the osmotic pressure of a solution. On a more simplistic level, osmolarity may be seen as a sum of all components in a solution that are capable of driving osmosis. To minimize or effectively eliminate transfer of solutes between a fluid in the PC and blood, through the peritoneal membrane, the fluid should have an osmolarity similar to that of blood plasma, typically in the range of range of 250-350 mOsm/l, for example in the range of 280-320 mOsm/l. The osmolarity of the test fluid F2 may be set to either minimize transfer of solutes in relation to the test fluid F2 as such or in relation to the mixture F3 that is generated when the test fluid F2 has been mixed with the modified first fluid F1′ (cf. stages IV and V in FIG. 4). It may be noted that spent PD fluid typically has consumed its ability of driving osmosis across the peritoneal membrane and thus prevents transfer of solvents through the peritoneal membrane. Thus, if the modified first fluid F1′ is generated by a dwell phase and thus corresponds to spent PD fluid, transfer of solids may be minimized or effectively eliminated by setting the osmolarity of the test fluid F2 similar to that of blood plasma.

One reason for setting the osmolarity of the test fluid F2 is to reduce ultrafiltration through the peritoneal membrane, since ultrafiltration may impair the accuracy of the calculated IPV1. This is in stark contrast to the prior art technique in which PD fluid is infused for mixing with spent PD fluid inside the PC, since PD fluid is by definition designed to promote transfer of fluid across the peritoneal membrane.

Reverting to the method 300 in FIG. 3, the first amount V1 and the second amount V2 may be set to improve accuracy, reduce the duration of the method 300, or reduce the risk for the patient. If step 302 is performed after a dwell phase and the PC is full of spent PD fluid (cf. stage II in FIG. 4), the first amount V1 needs to be large enough to make room for infusion of the second amount V2 of the test fluid F2 into the PC. If V1 is small, V2 also needs to be small. Thereby, C1 of the modified first fluid F1′ may be similar to C3 of the resulting mixture F3, causing the determination in step 307 to be highly sensitive to measurement errors in C1 and C3. Further, if C1 is measured by pumping extracted fluid through a sensor, V1 may need to be large enough to provide a reliable measurement value. If V1 is too large, leaving only a small amount of residual fluid IPV1 in the PC, the impact of measurement errors may again be significant. The second amount V2 needs to be large enough to result in a sufficient difference between C1 and C3. At the same time, a larger V2 increases the duration of the method 300, since V2 both needs to be infused into and drained from the PC. Also, a larger V2 will increase time that the patient is subjected to the test fluid.

In some embodiments, V1 is approximately 25%-95%, or approximately 70%-90% of the total amount of fluid present in the PC when step 302 is initiated, and V2 is approximately 25%-100%, or possibly approximately 40%-60%, of V1. As noted above with reference to the method 300 in FIG. 3, the dwell phase of step 301B may be a regular dwell phase of on-going PD therapy. The extraction of fluid in step 302 may also be performed as part of on-going PD therapy, specifically as part of a regular drain phase after the regular dwell phase. The regular drain phase may be interrupted at any selected time point, whereupon the test fluid is supplied in accordance with step 304. The regular drain phase is then resumed when steps 305-306, and optionally step 307, have been completed. The selected time point may be adjusted to result in a desired first amount (V1) being extracted from the PC. It is even conceivable that step 304 is performed when the regular drain phase is completed, assuming that a sufficient amount of fluid is left in the PC at this time.

In some embodiments, the APD system is configured to provide the first fluid as treatment fluid in a first fluid exchange cycle, and to provide the second fluid as treatment fluid in a second, consecutive fluid exchange cycle. In such an APD system, the method 300 in FIG. 3 may be performed without further modification of the PD session. Steps 301A, 301B, 302 may correspond to the fill phase, dwell phase and drain phase, respectively, of the first cycle. Step 303 may be performed during the drain phase of the first cycle. Steps 304, 305 may correspond to the fill phase and the drain phase, respectively, of the second cycle, and step 306 may be performed during the drain phase of the second cycle. Thus, by appropriately timing the measurement of CRP values during the regular phases of such a PD session, IPV1 may be determined in accordance with step 307.

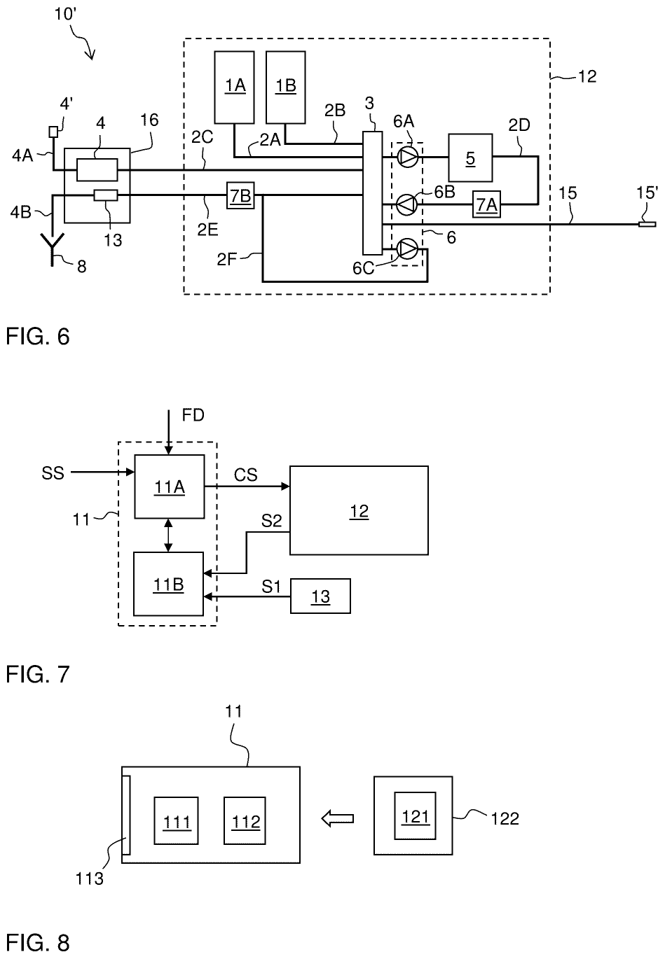

While the method 300 may be implemented on any type of PD system, it will be described with reference to an example given in FIGS. 6-7. FIG. 6 shows a fluid system 10′ that may be part of the PD system 10 in FIG. 1, and FIG. 7 schematically illustrates an installation for control of the PD system 10 in FIG. 1. It should be emphasized that FIGS. 6-7 are merely given as a non-limiting example and that many variations are conceivable without deviating from the principles of the technique described herein.

The fluid system 10′ in FIG. 6 comprises a water preparation device, WPD 16, which is configured to provide purified water to a fluid supply arrangement, FSA 12. The WPD 16 comprises an inlet tubing 4A with a terminal connector 4′ for connection to a tap water source (not shown). The tap water source may be a permanent outlet (water tap, faucet, spigot, etc.) or a tank, which is manually or automatically replenished with tap water as needed. A water purification unit 4 is configured to receive and process the tap water for purification. As used herein, “purification” refers to a process of substantially removing undesirable chemicals, biological contaminants, suspended solids, and gases from water, for the purpose of providing water with an acceptable purity for use in PD fluid. Purification may or may not involve sterilization. Numerous purification techniques are readily available for use in the water purification unit 4. The purified water is provided to the FSA 12 on a fluid line 2C (“water line”). In the illustrated example, the WPD 16 is further connected to receive outlet fluid from the FSA 12 on a fluid line 2E (“outlet line”). Depending on operating phase and implementation of the fluid system 10′, the outlet fluid may be any fluid described hereinabove, for example the first fluid (PD fluid) F1, the modified first fluid F1′, the test fluid F2, or the mixture F3. The WPD 16 comprises an outlet tubing 4B for directing the outlet fluid to a drain 8, as shown, or to a receptacle. A sensor arrangement 13 is disposed in the WPD 16 to measure the CRP of the outlet fluid.

In the illustrated example, the FSA 12 is configured to generate the PD fluid and the test fluid by mixing one or more concentrates with purified water. In FIG. 6, the FSA 12 comprises two containers 1A, 1B that hold a respective concentrate. Here, it is assumed that the respective concentrate is a liquid, although it is equally possible to use a concentrate in the form of a powder, which is being dissolved in purified water by the FSA 12. The containers 1A, 1B are connected by a respective fluid line 2A, 2B to a valve arrangement 3. The valve arrangement 3 is operable to selectively, based on one or more control signals from the control arrangement 11 (FIG. 1), establish fluid communication between different fluid lines in the FSA 12. The valve arrangement 3 may comprise any number and type of controllable valves. The valve arrangement 3 is also fluidly connected to the water line 2C and to the outlet line 2E. The valve arrangement 3 is further fluidly connected to a supply line 2D and a feed line 2F, as well as to the fluid connection device 15, which extends to a terminal connector 15′ for connection to the above-mentioned implanted catheter. The supply line 2D comprises a mixing section 5 and a flow meter 7A. The mixing section 5 is configured to ensure mixing of the concentrates with the purified water and may also be configured to performing further conditioning of the generated fluid, for example temperature adjustment and degassing. The feed line 2F connects to the outlet line 2E at a juncture, and a flow meter 7B is disposed in the outlet line 2E between the juncture and the WPD 16. The FSA 12 further comprises a pumping arrangement 6, which is configured to control the flow of fluids through the FSA 12, for example when generating and supplying fluid to the PC and when extracting fluid from the PC. In the illustrated example, the pumping arrangement 6 comprises three pumps 6A, 6B, 6C. Pumps 6A, 6B are arranged in the supply line 2D upstream and downstream of the mixing section 5, and pump 6C is arranged in the feed line 2F.

The FSA 12 may be configured to generate PD fluid and test fluid either batchwise or in-line. In batchwise generation, the mixing section 5 is configured to generate PD fluid and test fluid in batches and store them in reservoirs (not shown), from which the respective fluid is supplied by pump 6B along fluid line 2D as needed. The batchwise generation may be performed by operating the valve arrangement 3 to fluidly connect fluid lines 2A, 2B, 2C to fluid line 2D, for example in sequence, and by operating pump 6A to meter purified water and concentrates into mixing section 5 in accordance with a proportioning scheme for the PD fluid and the test fluid, respectively. In in-line generation, the mixing section 5 is configured to generate PD fluid and test fluid on-demand and without intermediate storage. The in-line generation may be performed similar to the batchwise generation, although the concentrates and the purified water are concurrently metered into the mixing section 5 to form the PD fluid or the test fluid. The skilled person understands that additional equipment may be needed in the FSA 12 to ensure adequate proportioning of the concentrates and the purified water. Further, in in-line generation, pump 6B may be omitted since the flow of PD fluid or test fluid may be driven through the mixing section 5 by pump 6A.

The fluid system 10′ in FIG. 6 is selectively operable in a first supply phase, in which PD fluid is pumped through the fluid connection device 15 into the PC, and a second supply phase, in which test fluid is pumped through the fluid connection device 15 into the PC. The fluid system 10′ in FIG. 6 is further selectively operable in an extraction phase, in which fluid is pumped through the connection device 15 from the PC to the drain 8. The extraction phase may also involve measurement of the CRP by the sensor arrangement 13. The fluid system 10′ may also be operable in a measurement phase in which the test fluid is pumped through the sensor arrangement 13 to the drain 8. The skilled person understands that the flow of fluid through the fluid system 10′ in the different phases is controlled by the control arrangement 11 (FIG. 1) providing dedicated control signals to the valve arrangement 3 and the pumping arrangement 6.

Turning to FIG. 7, the PD system 10 may be operated by a control arrangement 11, which comprises the control unit 11A and the calculation unit 11B. The control unit 11A is configured to control the operation of the fluid system 10′, here represented by the FSA 12. The control unit 11A is configured to generate dedicated control signals CS for components in the FSA 12, such as the valve arrangement 3 and the pumping arrangement 6 (FIG. 6). As indicated, the control unit 11A may generate CS based on settings SS and feedback data FD. The settings SS may be obtained from an internal memory of the FD system 10 and/or entered by a user via the UI 14 (FIG. 1). The feedback data FD is implementation-specific and will not be described in detail herein. In the specific example of FIG. 6, the feedback data FD may comprise data generated by the flow meters 7A, 7B.

The calculation unit 11B is configured to determine the IPV at one or more time points during PD therapy. As shown, the calculation unit 11B is connected to receive first sensor data S1 from the sensor arrangement 13, and second sensor data S2 from the FSA 12. The first sensor data S1 comprises CRP values, and the second sensor data S2 is representative of the amount of test fluid (V2) supplied to the PC by the FSA 12. In the example of FIG. 6, the second sensor data S2 may be generated by the flow meter 7A. In a variant, if the pump 6A (in-line generation) or the pump 6B (batchwise generation) is a volumetric pump with a known stroke volume, the second sensor data S2 may be indicative of the number of pumping strokes performed by the pump 6A, 6B when pumping the test fluid into the PC, thereby enabling volumetric calculation of the pumped amount of test fluid (“volumetric pumping”). As indicated by a double-ended arrow, the control unit 11A and the calculation unit 11B may also exchange data. For example, the calculation unit 11B may derive timing information about different operating phases of the FD system 10 from the control unit 11A, so as to allow the calculation unit 11B to timely obtain measurement values from the sensor data S1, S2.

In another example, the control unit 11A may request the calculation unit 11B to calculate the IPV at a specific time point and then adjust its operation based on the thus-calculated IPV.

The control unit 11A and the calculation unit 11B need not be implemented as separate entities, as shown, but may instead be combined into a single unit in the control arrangement 11.

Generally, the control arrangement 11 may be implemented by hardware or a combination of software and hardware. In some embodiments, the hardware comprises one or more software-controlled computer resources. For example, as shown in FIG. 8, the control arrangement 11 may comprise one or more processors 111, memory 112, and an interface or circuit 113 for input and output of data. The interface 113 may be configured for wired and/or wireless communication. The memory 112 may include one or more computer-readable storage mediums, such as high-speed random access memory, and/or non-volatile memory, such as one or more magnetic disk storage devices, flash memory devices, or other non-volatile solid-state memory devices. The processor(s) 111 may, for example, include one or more of a CPU (“Central Processing Unit”), a DSP (“Digital Signal Processor”), a GPU (“Graphics Processing Unit”), a microprocessor, a microcontroller, an ASIC (“Application-Specific Integrated Circuit”), a combination of discrete analog and/or digital components, or some other programmable logical device, such as an FPGA (“Field Programmable Gate Array”). A control program 121 comprising computer instructions may be stored in the computer memory 112 and executed by the processor(s) 111 to implement logic that performs any of the methods, procedures, functions or steps described herein. The control program 121 may be supplied to the control arrangement 11 on a computer-readable medium 122, which may be a tangible (non-transitory) product (e.g. magnetic medium, optical disk, read-only memory, flash memory, etc.) or a propagating signal.

The method 300 may be performed by the control arrangement 11 in FIG. 7. Specifically, steps 301A, 301B, 302-306 and 308 may be performed by the control unit 11A, and steps 307 and 309 may be performed by the calculation unit 11B. In step 301A, the fluid system 10′ is operated in the first supply phase, to supply PD fluid from the mixing section 5 along fluid line 2D and the fluid connection device 15′ into the PC. The amount of infused PD fluid in step 301A may be monitored by use of the flow meter 7A, by volumetric pumping, etc. In optional step 301B, the fluid system 10′ is operated to perform a dwell phase. In step 302, the fluid system 10′ is operated in the extraction phase, to draw the first amount (V1) of fluid from the PC via the fluid connection device 15′, the feed line 2F and the outlet line 2E to drain 8. The extracted fluid thereby passes the sensor arrangement 13, which is operated to measure C1 (step 303). The amount of extracted fluid in step 302 may be monitored by use of the flow meter 7B, by volumetric pumping, etc. In step 304B, the fluid system 10′ is operated in the second supply phase, to supply the second amount (V2) of the test fluid from the mixing section 5 along fluid line 2D and the fluid connection device 15′ into the PC. The amount of infused test fluid in step 304B may be monitored by use of the flow meter 7A, by volumetric pumping, etc. Optionally, before step 304B, the fluid system 10′ may be operated in the measurement phase, to pump the test fluid from the mixing section 5 along fluid line 2D and outlet line 2E to drain, while the sensor arrangement is operated to measure C2. In step 305, the fluid system 10′ is operated in the extraction phase, to draw the third amount (V3) of fluid from the PC via the fluid connection device 15′, the feed line 2F and the outlet line 2E to drain 8. The extracted fluid thereby passes the sensor arrangement 13, which is operated to measure C3 (step 306). The amount of extracted fluid in step 303 may be monitored by use of the flow meter 7B, by volumetric pumping, etc. In step 307, IPV1 is calculated based on V2, C1, C2 and C3. When extraction of V3 is completed, either deterministically or dynamically in accordance with step 308, fluid system 10′ may be operated to perform a fill phase, as shown at stage VIII in FIG. 4, by supplying PD fluid from the mixing section 5 along fluid line 2D and the fluid connection device 15′ into the PC.

It is to be understood that the fluid system 10′ may comprise any number of different concentrates, which are mixed to generate the PD fluid and/or the test fluid. Other variants for generating the PD fluid and/or the test fluid have been described above and will not be reiterated. To implement step 304A, the fluid system 10′ is re-configured to supply the test fluid as a ready-made fluid, which may be held in any of the containers 1A, 1B in FIG. 6. In fact, both the PD fluid and the test fluid may be supplied as ready-made fluids. If the fluid system 10′ is exclusively configured to use ready-made fluids, the WPD 16 is omitted.

The sensor arrangement 13 need not be located in the WPD 16 but may instead be part of the FSA 12, for example adjacent to the flow meter 7B. In another example, the sensor arrangement 13 comprises sensor units that are distributed within the FSA 12. For example, the sensor arrangement 13 may comprise one sensor unit in the fluid line 2D downstream of mixing section 5, for measuring C2, and one sensor unit in the feed line 2F, for measuring C1 and C3. However, it may be desirable to use one and the same sensor unit in the sensor arrangement 13 for measuring C1 and C3, and C2 if measured. This may improve accuracy, by eliminating systematic errors, and/or reduce the need for calibration of separate sensor units.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1-20. (canceled)

21: A system for peritoneal dialysis, the system comprising:
a fluid supply arrangement operable to convey fluid to and from a peritoneal cavity,
a sensor arrangement operable to measure a concentration-related parameter, and
a control arrangement which is connected to the fluid supply arrangement and the sensor arrangement and configured to:
operate the fluid supply arrangement to supply a first fluid to the peritoneal cavity,
operate, after supplying the first fluid, the fluid supply arrangement to extract a first amount of fluid from the peritoneal cavity so as to leave an intraperitoneal amount of fluid in the peritoneal cavity,
obtain, from the sensor arrangement, a first value of a concentration-related parameter of the first amount of fluid,
operate the fluid supply arrangement to supply a second amount of a second fluid to the peritoneal cavity, the second fluid having a second value of the concentration-related parameter and forming a mixture with the intraperitoneal amount of fluid in the peritoneal cavity,
operate the fluid supply arrangement to extract a third amount of the mixture from the peritoneal cavity,
obtain, from the sensor arrangement, a third value of the concentration-related parameter of the third amount of the mixture, and
determine the intraperitoneal amount based on the second amount and the first, second and third values,
wherein the control arrangement is configured to operate the fluid supply arrangement so that the first and second fluids differ in composition.

22: The system of claim 21, which is operated to achieve a difference between the second value and the first value that exceeds a threshold value, which is set to achieve a predefined accuracy of the intraperitoneal amount as determined based on the second amount and the first, second and third values.

23: The system of claim 22, wherein the threshold value is set to correspond to the second value being about 20%-75% larger than the first value or about 20%-75% smaller than the first value.

24: The system of claim 22, wherein the fluid supply arrangement is operable to generate the second fluid on demand, and wherein the control arrangement is configured to, based on the first value, to operate the fluid supply arrangement to generate the second fluid to achieve the difference between the second value and the first value.

25: The system of claim 21, wherein the first fluid is a treatment fluid used in peritoneal dialysis therapy, and wherein the second fluid is a dedicated test fluid for use in determining the intraperitoneal amount.

26: The system of claim 21, wherein the second fluid and the first fluid differ in concentration of at least one solute that affects the concentration-related parameter.

27: The system of claim 21, wherein solutes in the second fluid are the same as solutes in the first fluid.

28: The system of claim 21, wherein the second fluid has a different concentration of at least sodium compared to the first fluid.

29: The system of claim 21, wherein the second fluid has a different concentration of an osmotic agent compared to the first fluid.

30: The system of claim 21, wherein the second fluid has an osmolarity to minimize transfer of solvents through a peritoneal membrane in the peritoneal cavity.

31: The system of claim 21, wherein the second fluid has an osmolarity of 250-350 mOsm/l.

32: The system of claim 21, wherein the second value is smaller than the first value.

33: The system of claim 21, wherein the control arrangement is configured to perform a sequence of fluid exchange cycles, each comprising a fill phase, a dwell phase and a drain phase, wherein the sequence of fluid exchange cycles comprises a fluid exchange cycle, in which the fluid supply arrangement is operated to supply the first fluid to the peritoneal cavity in the fill phase and extract the first amount of fluid in the drain phase, and a consecutive fluid exchange cycle, in which the fluid supply arrangement is operated to supply the second amount of the second fluid to the peritoneal cavity in the fill phase and extract the third amount of the mixture during the drain phase, and wherein the control arrangement is configured to obtain the first value during the drain phase of the first cycle and to obtain the third value during the drain phase of the second cycle.

34: The system of claim 21, wherein the control arrangement is configured to operate the fluid supply arrangement so that the first amount is 25%-95% of an estimated total amount of fluid present in the peritoneal cavity.

35: The system of claim 21, wherein the control arrangement is configured to operate the fluid supply arrangement so that the second amount is 25%-100% of the first amount.

36: The system of claim 21, wherein the control arrangement is configured to determine the intraperitoneal amount by use of a dilution formula, which is given by IPV1=V2·(C3−C2)/(C1−C3), wherein V2 is the second amount, C1 is the first value, C2 is the second value, and C3 is the third value.

37: The system of claim 21, wherein the control arrangement is configured to determine the intraperitoneal amount during extraction of the third amount and terminate the extraction of the third amount based on the determined intraperitoneal amount and the second amount.

38: The system of claim 37, wherein the control arrangement is configured to terminate the extraction of the third amount to attain a predefined difference between the third amount and a sum of the determined intraperitoneal amount and the second amount.

39: A computer-implemented method of operating a system for peritoneal dialysis, the method operating the system to:
supply a first fluid to a peritoneal cavity,
extract, after supplying the first fluid, a first amount of fluid from the peritoneal cavity so as to leave an intraperitoneal amount of fluid in the peritoneal cavity,
measure a first value of a concentration-related parameter of the first amount of fluid,
supply a second amount of a second fluid to the peritoneal cavity, the second fluid having a second value of the concentration-related parameter and forming a mixture with the intraperitoneal amount of fluid in peritoneal cavity,
extract a third amount of the mixture from the peritoneal cavity,
measure a third value of the concentration-related parameter of the third amount of the mixture, and
determine the intraperitoneal amount based on the second amount and the first, second and third values,
wherein the first and second fluids differ in composition.

40: A computer-readable medium comprising program instructions, which when executed by a processor in a system for peritoneal dialysis, causes the processor to:
supply a first fluid to a peritoneal cavity,
extract, after supplying the first fluid, a first amount of fluid from the peritoneal cavity so as to leave an intraperitoneal amount of fluid in the peritoneal cavity,
measure a first value of a concentration-related parameter of the first amount of fluid,
supply a second amount of a second fluid to the peritoneal cavity, the second fluid having a second value of the concentration-related parameter and forming a mixture with the intraperitoneal amount of fluid in peritoneal cavity,
extract a third amount of the mixture from the peritoneal cavity,
measure a third value of the concentration-related parameter of the third amount of the mixture, and
determine the intraperitoneal amount based on the second amount and the first, second and third values,
wherein the first and second fluids differ in composition.
